SEQUENCE LISTING (16 sequences)

SEQ ID NO: 1 - DNA, 98 bp, artificial
  FUE sequence
tgtgtgagtagttcccagataagggaattagggttcttatagggtttcgctcatgtgttgagcatataagaaacccttagtatgtatttgtatttgta

SEQ ID NO: 2 - DNA, 13 bp, artificial
  FUE sequence
tgtgtgagtagtt

SEQ ID NO: 3 - DNA, 22 bp, artificial
  FUE sequence
ttagtatgtatttgtatttgta

SEQ ID NO: 4 - DNA, 56 bp, artificial
  FUE sequence
tcatgtatctttttcacctgtgccttgtttttgcctgtgttccatgtcctactgtt

SEQ ID NO: 5 - DNA, 59 bp, artificial
  FUE sequence
tttgtgacgcgttaagttcctgtttttacagtattataagtacttgtgttctgacaatt

SEQ ID NO: 6 - DNA, 40 bp, artificial
  FUE sequence
tttatttgtgaaatttgtgatgctattgctttatttgtaa

SEQ ID NO: 7 - DNA, 56 bp, artificial
  FUE sequence
ccacttctttttgtcacttgaaaaacatgtaaaaataatgtactaggagacacttt

SEQ ID NO: 8 - DNA, 10 bp, artificial
  FUE sequence
ttctttttgt

SEQ ID NO: 9 - DNA, 18 bp, artificial
  FUE sequence
cagctgctttttgcctgt

SEQ ID NO: 10 - DNA, 55 bp, artificial
  FUE sequence
ttgacttgactcatgcttgtttcactttcacatggaatttcccagttatgaaatt

SEQ ID NO: 11 - DNA, 88 bp, artificial
  primer
attcggtttttaagaagatgcatgcctaacgtgttcttttttttttccaatgatttgtaatatacattttatgactggaaactttttt

SEQ ID NO: 12 - DNA, 33 bp, artificial
  primer
cccctccgcggtggtatgcacagctcagcactg

SEQ ID NO: 13 - DNA, 87 bp, artificial
  primer
gggaattcagagctcgtccttgtgatgatgatgatgatgaccagaagaagaaccgcgtggcacaaggttacgtatcttcattgtcat

SEQ ID NO: 14 - DNA, 19 bp, artificial
  primer
cccaagcgagaaccaagac

SEQ ID NO: 15 - DNA, 32 bp, artificial
  primer
cgcggatccgaattaatacgactcactatagg

SEQ ID NO: 16 - DNA, 21 bp, artificial
  primer
aagctccaagagaaaggcatc

(19) United States
(12) Patent Application Publication
(10) Pub. No.: US 2006/0168677 A1
(43) Pub. Date: Jul. 27, 2006

(54) Transcriptional termination of transgene expression using host genomic terminators

(75) Inventors: James Edward Brandle, London (CA); Kirk Devon Brown, London (CA)

Correspondence Address: KLARQUIST SPARKMAN, LLP, 121 SW SALMON STREET, SUITE 1600, PORTLAND, OR, 97204, US

(73) Assignee: Her Majesty The Queen in Right of Canada as Represented by the Min. of Agriculture & Agri-Food

(21) Appl. No.: 11/042,617

(22) Filed: Jan. 24, 2005

(51) Int. Cl. A01H 1/00; C12N 1/20; C12N 15/82; C12N 5/04; C12N 1/18

(52) U.S. Cl. 800/278; 800/294; 435/252.2; 435/254.2; 435/419; 435/468

(57) ABSTRACT

The present invention relates to a method for expressing a transgene in a host cell that permits transcriptional termination of the transgene to occur without having to rely on a functional termination site in the DNA used for the transformation. Additional 3′ regulatory sequences and end processing enhancing sequences and/or structures can be present in the transformation vector or as a fusion with the transgene of interest.

### BACKGROUND

The present invention relates to means for terminating transcription of a gene.

Organisms and cells are frequently transformed with genes to produce functional proteins of interest. This is accomplished by a number of methods including infection with bacteria (Agrobacterium tumafaciens or Agrobacterium rhizogenes) or viruses (PVX), particle bombardment, microinjection, liposome fusion, or the like.

All transformation methods described to date rely on expression cassettes consisting of naturally occurring or genetically engineered fusions of the transgene to expression elements intended to be functional in an operative association with the transgene in the subsequently transformed host. These expression elements include an upstream promoter to facilitate transcription and a downstream termination signal to facilitate termination of transcription by RNA polymerase II and subsequent 3′ end formation of the transcribed gene.

When expressing a transgene, it is understood that as a matter of course, a transformation vector should be used that contains a transgene operably linked to an upstream promoter and a downstream terminator. For example, in Agrobacterium-mediated transformation, the conventional T-DNA transformation vector contains all of the nucleotide sequence elements deemed necessary for transcription and subsequent expression of a transgene once the expression construct is integrated as a contiguous linear unit into the genome of the host cell. These sequences include a transgene operatively associated with both a 5′ promoter region, either native to the transgene or another promoter functional in the host to facilitate transcription of the transgene, and a 3′ terminator region either native to the transgene or one that is functional in the host. The function of the terminator region in the conventional vector is to preclude transcriptional read-through to neighbouring DNA by terminating the transcription of the transgene and facilitating subsequent polyadenylation of the cleaved transcript that is necessary for mRNA stability, transport and efficient expression of the transcript in the cytoplasm by the ribosomes.

A limitation of the conventional transgene expression system is the inherent constraint on transgene expression due to the invariant nature of the nucleotide sequence elements integrated concommitantly and operatively associated with the gene as an integral part of the expression construct. Thus, the range of expression properties and regulatory features of a transgene is limited by a method of transformation which relies on regulatory functions of a 3′ UTR (untranslated region) or functional elements contained within the transformation vector.

In order to optimize expression constructs for the effects of different 3′ UTR DNA sequences or termination signals on transgene expression by conventional methods requires the construction of unique expression cassettes for each combination of elements to be tested. In addition several independent transformation experiments are necessary to generate transgenic plants containing all combinations of the desired elements. A priori knowledge of the DNA sequence of the 3′ UTR or other functional element to be tested is also essential. Although genomic sequences for some species are becoming increasingly available there are still many species in which there is limited genome and gene sequence available. Furthermore, in those species where complete genome sequences are available, identification and annotation of 3′ UTRs is limited. The utility of 3′ UTR or other DNA sequence elements as modulators of gene expression can only be determined by a functional analysis of the sequence in the species of interest.

Gene silencing adds an additional limitation to transgene expression using conventional constructs. Gene silencing is characterized by small double stranded silencing RNA (siRNA) produced by the host cell that contain homology to the mRNA of introduced genes and has the effect of silencing expression. All elements of the expression construct including the terminator are subject to the silencing phenomena as in addition to genes of interest siRNA has also been detected with homology to promoters and importantly the nos terminator (Canto et al. 2002. Mol. Plant Microbe Interactions 15:1137-1146). siRNA toward the nos terminator has recently been implicated as a major determinant of the systemic nature of the gene silencing phenomenon emphazing the need for a method of transformation with less reliance on exogenously introduced sequences and particularly terminators to facilitate gene expression.

Finally, increasing public concern over the use of non-host or superfluous DNA sequences in the development of transgenic organisms carrying a wide range of traits useful to agriculture, medicine and industry has led to a need to minimize the overall amount of genetic information that is transferred to the host.

U.S. Pat. No. 5,045,461 describes a method of increasing nodulation of a plant capable of being nodulated by Bradyrhizobium sp. (Parasponis). The method comprises infecting such a plant with a Bradyrhizobium sp. (Parasponia) species mutated such that nodK is non-functional. Insertion mutations were constructed in nodK with a terminatorless kanamycin resistance cassette to allow, in principle, mutation of single genes in an operon by insertional inactivation without polar effects on the transcription of “downstream” genes in the operon since transcription would not be terminated by the insertion.

U.S. Pat. No. 5,436,392 patent relates to expression of an insect serine protease inhibitor (PI) in transgenic plants. Some constructs are made with and some without the 19S terminator. It is noted that although some of the constructs are without the 19S terminator, all the constructs in fact contain a terminator site which is essentially the endogenous terminator from the insect in which the PI cDNA was isolated. See the examples section 4 at column 10 lines 23-30 describing the cDNA for PI at SEQ ID No:1 and FIG. 3 as having a consensus polyadenylation signal AATAAA at position 1414.

Yamamoto et al. 2003. Plant J. 35:273-283 is based on the concept of endogenous gene tagging or trapping for the purpose of cloning the endogenous gene, disrupting its function, or assessing its upstream regulatory components such as the associated promoter. Yamamoto et al. describes three cassettes that include NptII but do not contain the NOS terminator (constructs yy323, yy327 and yy376). Inspection of the sequences of these constructs available in Genbank (Acc. nos. AB086435 and AB086436) reveal that although these do not contain the NOS terminator (tNOS), they do contain potential terminator sites between the stop codon of the NptII selectable marker and the left border of the vector. In yy327 (GenBank Accession no. AB086435) and similarly yy323, there are two potential poly A sites at position 3395-3400 (ATTAAA) and 3453-3458 (AATATA), the latter as part of the left border sequence. In yy376 (GenBank Accession no. AB086436) there is a potential poly A site at 3466-3471 (AATATA) which is part of the consensus of the left border.

The potential terminator sites in yy323 and yy327 are functional terminator sites; this is evident from the experimental outcome of Yamamoto's gene trap strategy. Yamamoto et al. intended to select for integrations of their T-DNA into endogenous genes to study regulation of expression of the trapped gene. The strategy uses in part a poly A trap such that only when the T-DNA has integrated into an endogenous gene will the selectable marker be expressed as a result of transcriptional fusion with the last exon of an endogenous gene. Accordingly when the authors used a cassette with no nos terminator instead of one with a nos terminator, they expected a decrease in the number of transgenic plants generated since their strategy predicts this. However, the expected outcome did not occur. This is most likely explained by the construct yy323 containing termination sites (identified above) of which the authors were unaware.

### SUMMARY OF THE INVENTION

The present invention relates to a method for expressing a transgene in a host cell that permits transcriptional termination of the transgene to occur without having to rely on a functional termination site in the DNA used for the transformation. Additional 3′ regulatory sequences and 3′ end processing enhancing sequences and/or structures can be present in the transformation vector or as a fusion with the transgene of interest. They comprise one or several heterologous far upstream transcription termination enhancer (FUE) sequences, or one or more additional copies of FUE sequences endogenous to the transgene of interest.

The method of the invention results in transcriptional fusion between the expression cassette containing the transgene and the genome of the host. The resulting transcript contains genomic sequence between the 3′ end of the integrated expression cassette until the point at which a functional host terminator is encountered and transcription read-through is terminated.

In an exemplified embodiment, an integrated T-DNA from a binary vector of the invention carrying a transgene of interest is, as result of read through transcription through the transgene of interest, operably associated with host encoded polyadenylation signals near the integration site. Thus in some embodiments, the invention provides a method of transformation and compositions comprising such binary vectors, their nucleotide sequences, genes of interest produced by such method and vectors, and cells comprising such vectors and their integrated sequences. The invention also provides methods for using such binary vectors for expressing genes of interest in host cells and organisms. By incorporating one or more heterologous FUE sequences, or one or more additional copies of endogenous FUE sequences into such binary vectors, recognition and transcriptional termination efficiency of host-encoded polyadenylation signals may be enhanced.

The transformation method of the invention provides improvements over conventional methods. Such improvements include a significant reduction in the quantity of non-host foreign DNA that must be introduced into the host cell to facilitate the expression of genes of interest. The method confers the ability to simultaneously generate with a single transformation vector host cells that display differential expression and regulation of the transgene of interest. It can also be used in conjunction with a high throughput functional screen for endogenous genomic sequences or structures that can function to confer expression characteristics to genes of interest. While not intending to be limited to any theory, it is believed that by allowing transcription read through to genomic sequences next to the integration site and facilitating the acquisition by transcriptional fusion of host-encoded DNA sequences to the 3′ end of the transcribed transgene of interest that these acquired DNA sequences will function to regulate transgene expression including termination of transcription of the gene.

In one aspect, the invention relates to an expression cassette comprising a promoter operably linked to a transgene, such that when the expression cassette is integrated in a host cell and the transgene is transcribed, transcription terminates at a non-coding region in the genome of the host cell and not at a sequence within the cassette.

In certain embodiments, when the transgene is transcribed, the resulting RNA transcript comprises non-coding sequence from the host cell at the 3′ end, and the cassette-derived sequence in the RNA transcript is contiguous at the 3′ end with the non-coding sequence from the host cell.

In one aspect, the host cell or organism is a eukaryotic cell and preferably a plant cell including dicots and monocots. The organism may also be an animal, fungus or yeast.

The non-coding region of the genome at which transcription terminates may be an intergenic region of the genome, an intronic region of a gene within the genome, or a regulatory region of a gene within the genome.

In another aspect, the invention relates to the expression cassette as described above which is free of potential transcription termination site in the region 3′ of the transgene. The potential transcription termination sites may be those identified by the HC_PolyA program. The region 3′ of the transgene in the cassette may also be manually scanned. Potential transcription termination sites where the host cell is a plant cell may include the sequences:

AACAAA, AATAAA, AATAAC, AATAAG, AATAAT, AATACA,
AATAGA, AATATA, AATATT, AATTAA, ACTAAA, AGTAAA,
ATTAAA, CATAAA, GATAAA, GATTAA, AATGGA, AATGAA,
AATCAA, AAAAAA, AAGAAA, AATCAA and TATAAA.

The expression cassette may be scanned so that the region 3′ of the transgene is free of these potential transcription termination sites.

The transgene of the cassette may encode a recombinant protein which is other than a selectable marker or a reporter.

In another aspect, the invention relates to the expression cassette as described above which further comprises a far upstream enhancer (FUE) sequence 3′ of the transgene.

In another aspect, the invention relates to a transformation vector comprising the expression cassette as described above. The transformation vector may further comprise a selectable marker gene. In certain embodiments, the transformation vector described above is an Agrobacterium vector.

In another aspect, the invention relates to an organism having stably integrated in its genome the expression cassette described above.

In another aspect, the invention relates to a method for expressing a transgene in a host cell, the method comprising the steps of: a) stably integrating into the host cell genome an expression cassette comprising a promoter functional in the host cell operably linked to a transgene; and b) culturing the host cell comprising the expression cassette under conditions suitable for expression of the transgene such that, when the transgene is transcribed, transcription terminates at a non-coding region in the host cell genome and not at a sequence within the cassette. In step (a), the expression cassette may be integrated in a non-coding region of the host cell.

In another aspect, the invention relates to the method as described above such that, when the transgene is transcribed, the resulting RNA transcript comprises non-coding sequence from the host cell at the 3′ end, and the cassette-derived sequence in the RNA transcript is contiguous at the 3′ end with the non-coding sequence from the host cell.

In another aspect, the invention relates to the method as described above wherein the expression cassette is free of potential transcription termination site in the region 3′ of the transgene.

In another aspect, the invention relates to the method as described above wherein the expression cassette is free of potential transcription termination site in the region 3′ of the transgene. The potential transcription termination sites may be those identified by the HC_PolyA program. The region 3′ of the transgene in the cassette may also be manually scanned. Potential transcription termination sites where the host cell is a plant cell may include the sequences:

AACAAA, AATAAA, AATAAC, AATAAG, AATAAT, AATACA,
AATAGA, AATATA, AATATT, AATTAA, ACTAAA, AGTAAA,
ATTAAA, CATAAA, GATAAA, GATTAA, AATGGA, AATGAA,
AATCAA, AAAAAA, AAGAAA, AATCAA and TATAAA.

In another aspect, the invention relates to the method as described above wherein the non-coding region of the genome at which transcription terminates is an intergenic region of the genome, an intronic region of a gene within the genome, or a regulatory region of a gene within the genome.

In another aspect, the invention relates to the method as described above wherein the transgene encodes a recombinant protein which is other than a selectable marker or a reporter.

In another aspect, the invention relates to the method as described above wherein the expression cassette further comprises a far upstream enhancer (FUE) sequence 3′ of the transgene.

In another aspect, the invention relates to a method for expressing a transgene in a host cell, the method comprising the steps of: a) transforming the host cell with the transformation vector as described above such that the expression cassette is stably integrated into the host cell genome; and b) culturing the host cell obtained from step (a) under conditions suitable for expression of the transgene such that, when the transgene is transcribed, transcription terminates at a non-coding region in the host cell genome and not at a sequence within the cassette.

In another aspect, the invention relates to the method as described above wherein the host cell is a plant cell (dicot or monocot), a fungal cell such as a yeast cell, or an animal cell.

In another aspect, the invention relates to a commercial package comprising the transformation vector as described above in a container, and written instructions for using the vector in integrative transformation of a host.

### BRIEF DESCRIPTION OF DRAWINGS OF EMBODIMENTS

FIG. 1A shows the pHosT transformation vector containing the IL-10 open reading frame (ORF) downstream of the 35S promoter and tCUP translational enhancer oriented toward the right border (RB). FIG. 1B shows a simplified model of expression cassette design illustrating orientation and direction of transcription of the gene of interest (GOI) toward the right border and into host genomic sequence. FIG. 1C shows the addition of far upstream enhancer sequences (FUE) to the expression cassette 3′ of the GOI and adjacent to the RB to enhance the efficiency of poly A site recognition and processing. “PRO” represents a promoter; “Ter” represents a Terminator; “Marker” represents a marker or selection gene.

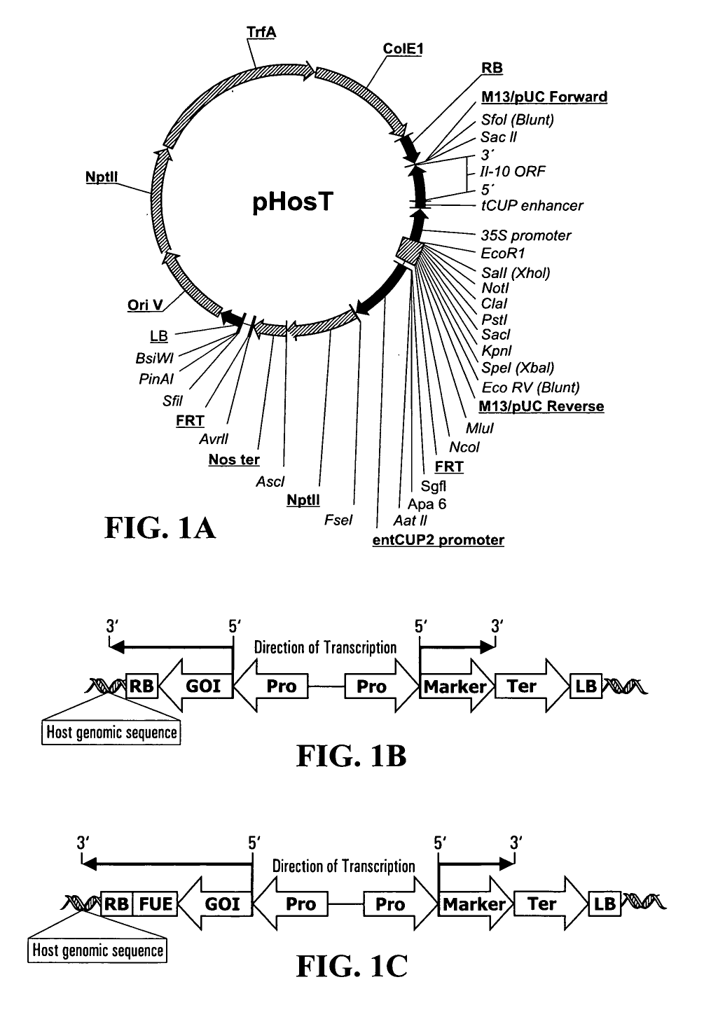

FIG. 2 shows expression of IL-10 protein in 19 tobacco transformants as evaluated by ELISA. IL-10 concentration was normalized to protein concentration as determined by Biorad assays performed on identical extract preparations.

FIG. 3A shows the sequence of the partial 3′ RACE product for Plant 14, a representative IL-10 expressing transformed plant. The sequence is written 5′ to 3′ and represents the IL-10 coding sequence (bold uppercase) followed by transcriptionally fused expression cassette sequence (uppercase) and genomic DNA (uppercase, enclosed in box), respectively. The putative poly A sites in the genomic DNA as identified by HC_POLYA are underlined with an asterisk indicating the poly A site within the accepted range of 10-40 base pairs upstream of the start of the poly A tail (lowercase). [Note the poly A tail is not part of the genomic sequence but is added as part of an enzymatic reaction catalyzed by poly A polymerase which results from recognition of the poly A site in the genomic sequence.]

FIG. 3B shows the results of the WU-BLAST 2.0 query of the tobacco genomic sequence from 3A against higher plant BACEND GSS (Genome Survey Sequences) verifying its tobacco genomic origin. Note that the WU-BLAST program from TAIR BLASTS sequences against GenBank GSS (genome survey sequences); this uses the same idea as the EST (expressed sequence tags) database with the exception that the sequences are genomic in origin as opposed to cDNA (mRNA) and are not likely to be exons.

### DETAILED DESCRIPTION OF EMBODIMENTS

The present invention relates to use of an expression cassette which allows a transgene of interest to acquire, via transcriptional fusion, host encoded termination sequences or other such structures. The sequences/structures become operatively associated transgene of interest and affect expression. The expression cassettes have no functional termination signals in order to allow transcription read through of the transgene of interest into host genomic DNA flanking the integration site. This allows the acquisition of host encoded regulatory sequences that includes, but is not limited to, termination sequences and structures.

The acquisition of host termination signals is achieved by read through transcription of the genetically integrated expression cassette into adjacent genomic DNA. If Agrobacterium-mediated transformation is used, the transgene of interest is specifically oriented within the transformation vector as close to the functional elements of the RB or LB of the T-DNA as possible so that the 3′ end of the transgene of interest is proximal to the border repeat and the promoter is proximal to the 5′ end of the transgene of interest.

In a preferred embodiment, the transgene of interest is oriented proximal to the RB. The process of T-DNA integration is polar, beginning at the RB. The RB end of the T-strand is protected from endonucleotyic degradation by covalent attachment of VirD2, which protects the integrity of the transgene of interest and allows the accurate prediction of the T-DNA end that will be integrated into the host genome. Although a similar process could be initiated at the LB, it is known that the LB is prone to incomplete nicking and vector DNA adjacent to it is often transferred during integration. Therefore, the genes in close proximity to the LB are prone to deletion events. Thus according to this scheme, if the T-DNA also contains a selection marker cassette in addition to the transgene cassette, transcription of the selection cassette would proceed in the opposite direction from that of the transgene cassette so that the transgene would be transcribed in the direction toward the right border and into genomic sequence next to the integration site.

In another embodiment, unnecessary vector sequence between the 3′ end of the transgene of interest (defined by the stop codon of the open reading frame) and the RB or LB sequence elements necessary for integration are removed from the vector. These elements become part of the 3′ UTR of the integrated transgene of interest via transcriptional fusion and may exert negative or unpredictable regulatory effects on gene expression. In another embodiment, potential termination sites are absent from either the transgene of interest or the vector sequence proximal to transgene of interest and the site of integration. Many variants of binary vectors contain residual termination signals from endogenous genes found in the native Ti plasmids. These signals can be identified by manual inspection or with computer software programs (i.e. HC_Poly A) and removed by site-directed mutagenesis to prevent premature termination preventing transcriptional read through into genomic sequence next to the integration site.

Transcription is initiated as a result of promoter activity and transcriptional read through of the transgene of interest proceeds from the site of initiation at the 5′ end of the gene through the open reading frame and through the remainder of vector sequence including the RB or LB that has become integrated as a process of the integration event along with the T-DNA into the host genome. The activity of the heterologous promoter may be constitutive, inducible or target cell-specific. Useful heterologous promoters include, but are not limited to 35S, tCUP and HPL.

The particular manner in which the expression cassette is integrated into the host genome is not critical to this invention and could be achieved by any of several established techniques including particle bombardment. However, with many of these techniques the site of integration of the expression construct in the host genome is an essentially random process which may limit the efficiency of the method. Recent studies have demonstrated that Agrobacterium mediated T-DNA integration displays a preference for areas of the genome in which termination signals and other regulatory sequences and structures are likely to reside. For example, T-DNA integration in Arabidopsis thaliana exhibits a preference for integration into AT rich components of the genome including 3′ UTRs, 5′ UTRs and promoters over introns and exons. of 88,120 T-DNA insertions characterized, 7.15% were found in 3′ UTRs and 36.7% were found in 3′ UTR, 5′ UtR and promoters (Alonso et al. 2003. Science 301:653-657). Therefore in the preferred embodiment of the invention Agrobacterium transformation is used.

The present invention is not limited to a particular Agrobacterium strain or Ti-plasmid, as it is known that the sequences of the imperfect repeats between Ti plasmids is highly conserved and border sequences from all Ti plasmids studied can function in heterologous Agrobacterium strains (Hellens et al. 2000. Trends Plant Sci. 5:446). The present invention anticipates improvements in the host range of species that are susceptible to Agrobacterium transformation. The manipulations of factors encoded on the Ti plasmid, the host bacterial chromosome or host factors may improve the host range or virulence of this system. For example, past modifications to the virulence of Agrobacterium has increased the transfer of T-DNA and its utility in the transformation of cereals by increasing the expression or activation state of virulence gene products including virG and virE1.

The invention can be used to transform any host cell including plants and yeast cells are transformed, as the efficiency of the method is enhanced by inherent genetic properties of these host genomes. Plants and yeast cells exhibit much less reliance on the strict mammalian consensus AATAAA sequence and much more heterogeneity in the types of sequences that can function as poly A signals. Thus one would expect an increase in the statistical frequency of encountering a functional termination sequence. In addition, polyploid plants provide an increased opportunity by virtue of genome size for the T-DNA to integrate into an area in which potential termination signals are likely to reside.

The cassettes and vectors of the invention may be beneficially used to express a transgene to produce any desired gene product in any host cell or organism. Accordingly, the vectors may additionally comprise one or more heterologous coding sequences, wherein such sequences are derived from sources other than the genome from which the vectors are derived. The product encoded by the transgene is also contemplated as preferably derived from sources other than the genome from which the vectors are derived.

In another embodiment, the heterologous coding sequences are each operably associated with an individual promoter to form expression cassettes, and such cassettes are inserted into binary vector T-DNA regions, preferably between the RB and LB. The expression cassettes may comprise promoters that are constitutive, inducible, tissue-specific, or cell-cycle specific. Examples of useful promoters include, but are not limited to CaMV, nos, ocs, tCUP and HPL.

Diverse gene products may be expressed using vectors of the invention. They include products derived from genomic DNA, cDNAs, synthetic genes, RNA, polypeptides, structural RNAs, anti-sense RNAs and ribozymes. In one embodiment, the vectors of the invention comprise and express one or more heterologous sequences encoding therapeutic polypeptides. Example therapeutic polypeptides include cytokines, growth factors, hormones, kinases, receptors, receptor ligands, enzymes, antibody polypeptides, transcription factors, blood factors, and artificial derivatives of any of the foregoing.

The invention also relates to a commercial package comprising the transformation vector as described herein in a container, with written instructions for using the vector in integrative transformation of a host. In equivalent embodiments, the commercial package comprises the transformation vector as described above, but wherein the vector does not already contain a transgene. Instead, the vector includes cloning sites to permit a transgene of interest to be inserted, and the kit's written instructions include directions for inserting the transgene into the vector.
(I) Definitions

“Endogenous cellular gene” refers to a gene that is native to a cell, which is in its normal genomic and chromatin context, and which is not heterologous to the cell.

“Endogenous gene” refers to a microbial or viral gene that is part of a naturally occurring microbial or viral genome in a microbially or virally infected cell. The microbial or viral genome can be extrachromosomal or integrated into the host chromosome. This term also encompasses endogenous cellular genes, as described above.

“Heterologous” is a relative term, which when used with reference to portions of a nucleic acid indicates that the nucleic acid comprises two or more subsequences that are not found in the same relationship to each other in nature. For instance, a nucleic acid that is recombinantly produced typically has two or more sequences from unrelated genes synthetically arranged to make a new functional nucleic acid, e.g., a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. In contrast, a naturally translocated piece of chromosome would not be considered heterologous in the context of this invention, as it comprises an endogenous nucleic acid sequence that is native to the mutated cell.

“Recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

“Reporter gene” refers to a nucleic acid that essentially encodes any gene product that can be expressed in the cell of interest and is assayable and detectable. The reporter gene must be sufficiently characterized such that it can be operably linked to the promoter. Reporter genes used in the art include the LacZ gene from E. coli, the CAT gene from bacteria, the luciferase gene from firefly, the GFP gene from jellyfish, galactose kinase (encoded by the galK gene), and beta-glucosidase (encoded by the gus gene).

“Promoter” refers to an array of nucleic acid control sequences that direct transcription. As used herein, a promoter typically includes nucleic acid sequences near the start site of transcription, such as, in the case of certain RNA polymerase II type promoters, a TATA element, enhancer, CCAAT box, SP-1 site, etc. As used herein, a promoter also optionally includes distal enhancer or repressor elements, which can be located as much as several thousand base pairs from the start site of transcription. The promoters often have an element that is responsive to transactivation by a DNA-binding moiety such as a polypeptide, e.g., a nuclear receptor, Gal4, the lac repressor and the like.

A “constitutive” promoter is a promoter that is active under most environmental and developmental conditions. An “inducible” promoter is a promoter that is active under certain environmental or developmental conditions.

“Operably linked” refers to a functional linkage between a nucleic acid expression control sequence (such as a promoter) and a second nucleic acid sequence, wherein the expression control sequence directs the extent of transcription of the second sequence.

An “expression cassette” is a transcription module comprising a nucleic acid to be transcribed (e.g. a transgene) operably linked to a promoter.

A “transformation vector” is a vehicle generated recombinantly or synthetically for deliverying a nucleic acid into a cell. It comprises a series of specified nucleic acid elements that permit integration and transcription of a particular nucleic acid in a host cell, and usually comprises elements for replication. Depending on the transformation method, the transformation vector may be a plasmid, virus, liposome, particles for bombardment etc. Typically, the transformation vector includes one or more expression cassettes. The term expression vector also encompasses naked DNA operably linked to a promoter.

“Transformation” refers to the introduction of nucleic acid into a recipient host. “Integrative transformation” refers to transformation where the introduced nucleic acid is integrated into the genome of the recipient.

By “host” is meant bacteria cells, fungi, animals or animal cells, plants or seeds, or any plant parts or tissues including plant cells, protoplasts, calli, roots, tubers, seeds, stems, leaves, seedlings, embryos, and pollen, that is capable to being transformed with a transformation vector and expression cassette. The host typically supports integration of the expression cassette. Host cells may be prokaryotic cells such as E. coli, or eukaryotic cells such as yeast, fungal, protozoal, higher plant (rice, tobacco, corn, Arabidopsis etc.), insect, amphibian cells, or mammalian cells such as CHO, HeLa, 293, COS-1, and the like, e.g., cultured cells (in vitro), explants and primary cultures (in vitro and ex vivo), and cells in vivo.

“Transgenic plant” refers to a plant where an introduced nucleic acid is stably introduced into a genome of the plant, for example, the nuclear or plastid genomes. A transgenic plant is produced by transformation of plant cells with a vector, including an expression cassette that comprises a transgene of interest, the regeneration of a population of plants resulting from the insertion of the transgene into the genome of the plant, and selection of a particular plant characterized by insertion into a particular genome location. The term transgenic plant also refers to the original transformant and progeny of the transformant that include the heterologous DNA. The term transgenic plant also refers to progeny produced by a sexual outcross between the transformant and another variety that include the heterologous DNA. Even after repeated back-crossing to a recurrent parent, the inserted DNA and flanking DNA from the transformed parent is present in the progeny of the cross at the same chromosomal location. The term transgenic plant also refers to DNA from the original transformant comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

“Expression” refers to the transcription of a gene to produce the corresponding mRNA and, if the mRNA is capable of being translated, translation of this mRNA to produce the corresponding gene product (i.e., a peptide, polypeptide, or protein).

“Expression of antisense RNA” refers to the transcription of a DNA to produce a first RNA molecule capable of hybridizing to a second RNA molecule. Formation of the RNA—RNA hybrid inhibits translation of the second RNA molecule to produce a gene product.

“Regulatory region” refers to a nucleotide region located upstream (5′), within, or downstream (3′) of a coding sequence in the genome. Transcription and expression of the coding sequence is typically impacted by the presence or absence of the regulatory sequence.

“Isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

“Non-coding region” refers to a segment of the genome that does not encode a polypeptide. A non-coding region includes intergenic regions (which are between genes), intronic and regulatory regions (which are within genes).

“Intergenic region” refers to DNA sequences located between genes and have no known function. These sequences are interspersed throughout the genome.

“Intronic region” refers to non-coding, intervening sequences of DNA that are transcribed, but are removed from within the primary gene transcript and degraded during maturation of messenger RNA; so it is a part of a gene outside an exon. Most genes in the nuclei of eukaryotes contain introns, as do mitochondrial and chloroplast genes.

“Transcription unit” refers to a region of DNA that transcribes a single primary transcript.

“Transgene” is a nucleic acid integrated into an organism. The organism may not have had the nucleic acid originally, or may have had a different version of the nucleic acid such as an allelic variant or multiple copies of the nucleic acid. Alternatively, the organism may have the same nucleic acid (i.e. an endogenous gene), but in that case, the transgene is operably linked to a heterologous promoter such that the combination of promoter and transgene does not occur in the organism originally. A transgene can encode a recombinant protein including fusion proteins, or an antisense RNA, or an RNAi sequence that interferes with expression of a target sequence, or can encode a gene product that affects a phenotypic trait such as cold tolerance (in a plant) etc.

“Transcription termination site” refers to a site in the DNA sequence which signal the termination of transcription. The site consists of a recognition element generally 8-31 base pairs upstream of a cut site.
(II) Termination

Transformation methods used to date rely on expression cassettes containing the transgene operably linked to expression elements intended to be functional with the transgene in the subsequently transformed host. In eukaryotes, these expression elements include a downstream termination site to facilitate termination of transcription by RNA polymerase II and subsequent 3′ end formation of the transcribed gene.

The core termination site, alternatively referred to as polyadenylation signal (poly A signal) or near upstream element (NUE) consists of a recognition element (the termination signal) generally 8-31 base pairs upstream of a consensus CA (mammals) or YA (plants) dinucleotide cleavage/polyadenylation cut site. In mammalian cells, the termination signal is a highly conserved AAUAAA hexanucleotide element whereas in plants and yeast the signals can deviate considerably from the mammalian consensus and may be composed of larger and more complex sequences (Li. 1995. Plant Mol. Biol. 28: 927).

The 3′ end of a transcribed gene, referred to as the 3′ untranslated region (3′ UTR), is composed of sequences or structures located between the stop codon, which signifies the end of translation, and the remainder of the transcribed mRNA, which includes the termination signal up to the cut site. Recognition of the termination signal by host encoded factors is followed by cleavage of the transcript at the cut site and the template-independent addition of an approximately 250-nucleotide poly(A) tail. A growing number of 3′ UTRs have been shown to contain sequence elements located upstream of the termination signal (NUE) that function to enhance recognition of the signal and increase the efficiency of mRNA 3′ end processing including transcription termination and polyadenylation.

Far upstream enhancers (FUEs) have been found in the 3′ UTRs of various viruses including cauliflower mosaic virus (Sanfacon et al. 1991. Genes & Dev. 5:141-149), ground squirrel hepatitis virus (Cherrington et al. 1992. J, Virol. 66:7589-7596), HIV-1 (Valsamakis et al. 1992. Mol. Cell. Biol. 12:3699-3705)(Gilmartin et al. 1992. EMBO J. 11:4419-4428), equine infectious anemia virus (Graveley et al. 1996. J. Virol. 70:1612-1617), simian virus 40 (SV40) (Carswell et al. 1989. Mol. Cell. Biol. 9:4248-4258), adenovirus (Prescott et al. 1994. Mol. Cell. Biol. 14:4682-4693; DeZazzo et al. 1989. Mol. Cell. Biol. 9:4951-4961); in mammalian genes including human complement C2 (Moreira et al. 1995. EMBO J. 14:3809-3819; Moreira et al. 1998. Genes & Dev. 12:2522-2534) and lamin B2 (Brackenridge et al. 1997. Nucleic Acid Res. 25:2326-2335); and in plant genes including pea rbcS (Bradley et al. 1992. Mol Cell. Biol. 12:5406-5414).

Sequences comprising the 3′ UTR including the termination signal are operably associated with the transcribed gene and can confer regulatory properties that influence gene expression. Addition of the poly (A) tail influences aspects of mRNA metabolism, such as stability, translational efficiency, and transport of processed mRNA from the nucleus to the cytoplasm.

Termination signals in plants can vary widely from the strict consensus AAUAAA found in mammals and can be larger and more complex thereby increasing the number of potential sequences which could become associated with the transgene of interest increasing the efficiency of the method. Saturation mutagenesis of the consensus AAUAAA in plants and yeast revealed that all single base pair mutations were recognized with up to 60% of wild-type efficiency (Rothnie et al. 1994. EMBO J 13:2200; Guo et al. 1995. Mol. Cell Biol. 15:5983). Further, it is known that the particular termination signal used by a transgene of interest can influence MRNA processing and expression thereby increasing the potential utility of the invention.

It has been found that even in mammals AAUAAA is not always optimal or function at all in a given context (Wu et al. 1994. Mol. Cell Biol. 14:6829; Sanfacon et al. 1994. Virology 198:39). Further, strong polyadenylation signals have been observed to increase the level of precursor cleavage and the length of poly (A) of mRNA produced in vitro (Lutz et al. 1996. Genes & Dev. 10:325-337) and increased poly (A) tail length has been correlated with enhanced transgene expression (Loeb et al. 1999. West Cost Retrovirus Meeting, abstract p57). Provided a termination signal is present somewhere in the vicinity of the integration site it is likely to function as such as the cut site has been found to be less critical. Numerous studies in which the cut site was removed or mutated have demonstrated that cleavage is still able to occur at an appropriate position downstream of the termination signal even in the absence of a suitable YA dinucleotide (Guerineau et al. 1991. Mol. Gen. Genet. 226:141-144; MacDonald et al. 1991. Nucleic Acid Res. 19:5575-5581; Merits et al. 1995. Virology 211:345-349; Mogen et al. 1992. Mol. Cell Biol. 12: 5406-5414)(Wu et al. 1993. Plant J. 4:535-544). Further, alteration of the termination signal can result in a change in the location of the cut site that is used (Wu et al. 1994. Mol. Cell Biol. 14:6829-6838).

The addition of Far Upstream Enhancer (FUEs) sequences to the transformation vector increase the efficiency at which endogenous potential termination signals are recognized and function efficiently as such. FUEs are generally found as functionally redundant elements within a 3′ UTR of a given transgene and can exert control over more than one termination signal. The functional conservation of these elements is indicated by the ability of the CaMV FUE to replace the FUE for zein, FMV, and rbsS-E9 and vice versa (Mogen et al. 1992. Mol Cell Biol 12:5406-5414; Sanfacon. 1994. Virology 198: 39-49; Wu et al. 1994. Mol. Cell Biol. 14: 6829-6838). The FUE of CaMV and FMV have also been demonstrated to augment each other (Sanfacon. 1994. Virology 198: 39-49).

Although FUE sequences are generally composed of U- or UG-rich and are functionally conserved and interchangeable across species, there is no clearly definable or unambiguous sequence homology among those identified to date. This functional conservation despite no obvious similarity in primary structure has led to the suggestion that a basic 3′ end processing machinery has been conserved between dicots and monocots as well as other organisms (Rothnie. 1996. Plant Mol. Biol. 32:43-61) and also demonstrates that the FUE sequence only affects the efficiency at which a given termination signal is utilized and does not determine the 3′ end profile of a given gene.

Heterologous FUEs have also been shown to induce processing of cryptic termination signals (i.e. signals not associated with a gene) when placed upstream of them (Rothnie et al. 1994. EMBO J. 13:2200-2210; Sanfacon. 1994. Virology 198:39-49; Sanfacon et al. 1991. Genes Dev 5:141-149). The CaMV FUE UUUGUA motif was able to induce the recognition of a cryptic site in the nos terminator in an additive an orientation dependent manner (Rothnie et al. 1994. EMBO J. 13:2200-2210). A compilation of FUE sequences from plant, animal and yeast sources mostly of viral origin reveal a loose consensus motif UUUGUA which has been shown to enhance 3′ end processing in an orientation and distance dependent manner, the effect of which was additive when present in tandem repeated copies upstream of a termination signal (Rothnie. 1996. Plant Mol. Biol. 32:43-61).

The FUE of the ground squirrel hepatitis virus also influences the activity of the core termination signal in an orientation-dependent, additive but distance-independent manner (Russnak. 1991. Nucleic Acid Res. 19:6449-6456). However, there are FUE sequences which do not contain this motif indicating that this may be only one of a class of FUE sequences with other consensus sequences that have yet to be identified. In addition it is likely that surrounding sequence context contribute to the interaction efficiency of a given FUE sequence with a particular termination signal.

The expression cassettes of the invention may contain sequences to enhance the recognition and efficiency of processing of host encoded termination sequences or structures, which may comprise one or several FUE sequences that become operably associated with an endogenous termination signal in the host genome. The FUE sequence may be a heterologous FUE sequence or an additional copy of any endogenous FUE sequence which may be present in the transgene of interest. In one embodiment, the expression cassette comprises one or several heterologous FUE sequences. In another embodiment, the expression cassette comprises one or several additional copies of an endogenous FUE sequence. In a further embodiment, the expression cassette comprises both heterologous and an additional copy of endogenous FUE sequences.

The vectors of the invention may additionally comprise a microbial origin of replication and a microbial screenable or selectable marker for use in amplifying vector sequences in microbial cells, such as bacteria and yeast.

The expression cassettes of the invention may comprise any FUE sequence or active segments thereof. Preferably, the FUE is from a viral or eukaryotic gene. Example viral FUEs include, but are not limited to, cauliflower mosaic virus, ground squirrel hepatitis virus (e.g. UGE), HIV-1 (e.g., UHE), SV40 virus (e.g., USE), or equine infectious anemia virus UE (see Figure). Examples of eukaryotic FUEs include, but are not limited to, those of mammalian complement C2 and lamin B2 genes.

Specific embodiments of FUEs and active FUE segments (i.e., FUE sequences collectively) that may comprise vectors of the invention include, but are not limited to, the following:
a) The cauliflower mosaic virus FUE comprising the sequence TGTGTGAGTAGTTCCCAGATAAGGGAATTAGGGTTCTTATAGGGTTTCGCTCAT GTGTTGAGCATATAAGAAACCCTTAGTATGTATTTGTATTTGTA (SEQ ID NO:1); and all active segments thereof. In preferred embodiments, such segments comprise the sequence TTGTA, TGTGTGAGTAGTT (SEQ ID NO:2), or TGTGTTG, or TTAGTATGTATTTGTATTTGTA (SEQ ID NO:3).
b) The ground squirrel hepatitis virus FUE (UGE) comprising the sequence TCATGTATCTTTTTCACCTGTGCCTTGTTTTTGCCTGTGTTCCATGTCCTACTGTT (SEQ ID NO:4); and all active segments thereof. In preferred embodiments such segments comprises the sequence TTTTT, or TTGTTTTTG, or TGTGTT.
c) The equine infectious anemia virus FUE comprising the sequence TTTGTGACGCGTTAAGTTCCTGTTTTTACAGTATTATAAGTACTTGTGTTCTGACAATT (SEQ ID NO:5); and all active segments thereof. In preferred embodiments, such segments comprise the sequence TTTGT, or TGTTTTT, or TTGTGTT.
d) The FUE from SV40 (USE) comprising the sequence TTTATTTGTGAAATTTGTGATGCTATTGCTTTATTTGTAA (SEQ ID NO:6); and all active segments thereof. In preferred embodiments, such segments comprise the sequence ATTTGTGA or ATTTGTAA.
e) The adenovirus L3 FUE comprising the sequence CCACTTCTTTTTGTCACTTGAAAAACATGTAAAAATAATGTACTAGGAGACACTTT (SEQ ID NO:7); and all active segments thereof. In preferred embodiments such segments comprises the sequence TTCTTTTTGT (SEQ ID NO:8).
f) The HIV-1 FUE (also known as UHE) comprising the sequence CAGCTGCTTTTTGCCTGT (SEQ ID NO:9); and all active segments thereof. In preferred embodiments such segments comprise the sequence TTTTT.
g) The complement C2 FUE comprising the sequence TTGACTTGACTCATGCTTGTTTCACTTTCACATGGAATTTCCCAGTTATGAAATT (SEQ ID NO: 10); and all active segments thereof. In preferred embodiments such segments comprise the sequence TTGTTT or GTTATG.
h) The lamin B2 FUE comprising the sequence ATTCGGTTTTTAAGAAGATGCATGCCTAACGTGTTCTTTTTTTTTTCCAATGATTT GTAATATACATTTTATGACTGGAAACTTTTTT (SEQ ID NO:11); and all active segments thereof. In preferred embodiments, such segments comprise the sequence TTTTT, or GTGTT, or TTTGT, or TTTTATG.

The expression cassettes of the invention may comprise one or several FUE sequences that become operably associated with the termination signals encoded by the host DNA once the transgene is inserted at the integration site. Specifically, the operable association refers to an incorporation of FUE sequence(s) that enhances the recognition, transcriptional termination activity and polyadenylation activity as a result of the host encoded signals. Expression cassettes containing these sequences may have various improved properties. Possible improvements include an increase in the sequence variability and absolute number of poly A signals that can be recognized as such in the host and an increase in the efficiency of RNA processing at recognized sites leading ultimately to the increased production of expression cassette encoded RNA and/or expression cassette encoded polypeptide; and higher transgene of interest expression in host cells.

A FUE sequence may become operably associated with the host encoded termination signal by having the FUE sequence inserted at a site in the expression cassette 5′ upstream of the signal and 3′ downstream of the transgene of interest. The orientation of the inserted FUE sequence to the termination signal may or may not be in the same orientation to the termination signal in the transgene from which the sequence was derived.

The invention contemplates expression cassettes comprising all possible combinations of multiple FUE sequences. Example combinations include, but are not limited to: two or more heterologous FUE sequences are identical or are derived from the same FUE; two or more heterologous FUE sequences that are derived from different FUEs; two or more copies of the same endogenous FUE sequence, two or more copies of different endogenous FUE sequences; one or more heterologous FUE sequence and one or more additional copies of an endogenous FUE sequence.

The transformation method of the invention provides numerous improvements over conventional methods including a significant reduction in the quantity of non-host foreign DNA that must be introduced into the host cell to facilitate the expression of genes of interest; the ability to simultaneously generate with a single transformation vector host cells that display differential expression and regulation of the transgene of interest and the use of the method as a high throughput functional screen for endogenous genomic sequences or structures that can function to confer expression characteristics to genes of interest.

The method of the invention does not require a priori knowledge of a 3′ UTR sequence or structure as preferential integration events in 3′ UTRs and other areas of the host genome that may confer expression elements allows identification by virtue of the qualitative and quantitative functional screen sequences or structures that can function as 3′ UTRs or expression elements for a transgene of interest in a host of interest. A simple screen of the transgenic plants for levels of transgene of interest expression allows a qualitative and quantitative functional test. Further, once an optimal level of expression has been identified (which may or may not be the highest expression level), one can determine by simple molecular biological methods the host 3′ sequences that confer the desired expression for further manipulation or downstream experimentation.

Unique founder plants with transgene of interest transcriptional chimeras with various 3′ UTR's and other regulatory elements conferring varying levels of transgene of interest expression can be created and identified in the same transformation procedure. While not intending to be limited to any theory, it is believed that by allowing transcription read through to genomic sequences next to the integration site and facilitating the acquisition by transcriptional fusion of host-encoded DNA sequences to the 3′ end of the transcribed transgene of interest that these acquired DNA sequences will function to terminate the transcription of the transgene and that the acquired 3′ UTR may lead to increases in the production, stability, nuclear export and/or translation of vector encoded mRNA, and that such increases may lead to higher vector encoded mRNA production and/or transgene expression, and hence higher transgene expression in host cells.

It is possible to search for predicted, possible termination signals. A program that may be used to predict potential termination sites is HC_POLYA which was developed as a component of a larger package of tools for the prediction and analysis of protein-coding gene structure. The HC_POLYA program is available at http://125.itba.mi.cnr.it/˜webgene/wwwHC polya.html.

The HC_POLYA program predicts the termination signas in the 3′ gene regions by applying the Hamming-Clustering network (HC) to the poly(A) signal determination in DNA sequences. This approach employs a technique deriving from the synthesis of digital networks in order to generate prototypes, or rules, which can be directly analysed or used for the construction of a final neural network. For HC_POLYA, more than 1000 poly-A signals have been extracted from EMBL database rel. 42 and used to build the training and the test set. See Milanesi et al. (1996) Comput. Applic. Biosci, 12 (5) p399-404 (1996); Milanesi et al. (1995) Recognition of Poly-A signals with Hamming Clustering. In: “Proceedings of the Third International Conference on Bioinformatics, Supercomputing and Complex Genome Analysis” (H. A. Lim, J. W. Fickett, C. R. Cantor and R. J. Robbins, eds.), World Scientific Publishing, Singapore, pp. 461-466; Milanesi and Rogozin. Prediction of human gene structure. In: Guide to Human Genome Computing (2nd ed.) (Ed. M. J. Bishop) Academic Press, Cambridge, 1998, 215-259.

We have used the HC_POLYA program to identify the number of potential termination sites in a variety of plant genomes (Arabidopsis, rice, corn and tomato). For example, for Arabidopsis thaliana in which randomly generated fragments representing ˜5.4% of the genome have been run through the program to predict potential poly A sites of length 6 on the direct and complement strand, the results indicate a ubiquitous distribution with an average distance of one predicted site every 86+/−23 bases on the direct strand and one site every 90+/−20 bases on the complement strand.

Results we obtained on the number of poly A sites in a number of different plant species are set forth in Table 1. We chose to represent the data as the average number of 6 base pair poly A sites as identified by HC_Poly A on either the direct or complement strand per kilobase of genomic DNA sequence with standard deviations. (the figure for both strands is the combined average).

It is also possible to search for predicted, possible termination signals manually. For plants, such signals include the sequences:

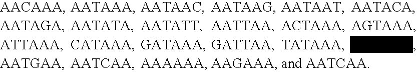

Where the invention is applied to plants, it is noted that integrative transformation may occur not just into the nuclear genome, buty also the plastid genome.

Methods to transform the plastid genomes of plants are known in the art and described in, for example U.S. Pat. No. 6,680,426, U.S. Pat. No. 6,642,053, US 20040177402, U.S. Pat. No. 6,515,206, U.S. Pat. No. 5,932,479, U.S. Pat. No. 5,877,402, U.S. Pat. No. 5,866,421, and U.S. Pat. No. 5,693,507.

We have also used the HC_POLYA program to identify the number of potential termination sites in a variety of plant chloroplast genomes. Results we obtained on the number of poly A sites in a number of different plant species (Arabidopsis, rice, corn, and tobacco) are also set forth in Table 1. The numbers closely approximate those found in the nuclear genome and indicate that the method of the invention as described above would also be functional in chloroplast transformation.

TABLE 1
The number of HC_POLYA predicted poly A sites on the
direct and complement strands of the nuclear and chloroplast
genomes of various species.

| | Direct | Complement | Direct | Complement |
|---|---|---|---|---|
| Species | (nuclear) | (nuclear) | (chloroplast) | (chloroplast) |
| Arabidopsis | 13.0 +/− 2.8 | 12.9 +/− 2.9 | 13.8 +/− 3.8 | 13.7 +/− 3.3 |
| thaliana | (3,224,000) | (3,224,000) | (154,478) | (154,478) |
| Oryza sativa | 9.5 +/− 2.0 | 9.5 +/− 2.0 | 10.7 +/− 1.3 | 10.8 +/− 2.1 |
| (rice) | (18,259,000) | (18,259,000) | (124,000) | (124,000) |
| Zea mays | 6.5 +/− 1.8 | 6.7 +/− 1.7 | 11.5 +/− 1.3 | 11.7 +/− 2.2 |
| (corn) | (3,016,407) | (3,016,407) | (124,000) | (124,000) |
| Lycopersicon | 15 +/− 1.5 | 15.6 +/− 1.7 | N/D | N/D |
| esculentum | (784,557) | (784,557) | | |
| (tomato) | | | | |
| Saccharomyces | 10.3 +/− 1.1 | 10.3 +/− 1.1 | N/A | N/A |
| cerevisiae | (858,700) | (855,600) | | |
| (yeast) | | | | |
| Asperigillus | 3.6 +/− 0.8 | 3.6 +/− 0.8 | N/A | N/A |
| nedulans | (424,700) | (424,700) | | |
| (fungi) | | | | |
| Pan | 10.1 +/− 3.4 | 10.2 +/− 3.3 | N/A | N/A |
| troglodytes | (6,138,000) | (6,107,000) | | |
| (chimpanzee) | | | | |
| Nicotiana | N/D | N/D | 11.5 +/− 2.3 | 11.7 +/− 2.3 |
| tabacum | | | (155,000) | (155,000) |
| (tobacco) | | | | |

The data is represented as the average number of 6 base poly A sites per kilobase of scanned DNA +/− the standard deviation.
The numbers in brackets represent the number of bases scanned.
N/D = Not Done;
N/A = Not Applicable.

According to the present invention, as a result of transcriptional read-through when the transgene is transcribed, the resulting RNA transcript may comprise at the 3′ end a non-coding sequence derived from the host cell. The cassette-derived sequence in the RNA transcript may be contiguous at the 3′ end with the host cell-derived non-coding sequence.

Whether transcriptional read-through of the transgene has occurred can be readily determined using methods known in the art. Common methods used to determine sequences of fusion transcripts include 3′ Rapid Amplification of cDNA Ends (RACE), cDNA cloning, and cloning of genomic DNA surrounding the site of transgene integration. Many commercial kits are available for RACE, e.g. the GeneRacer RLM-RACE kit from Invitrogen.

To determine whether or not transcriptional read-through of the transgene has occurred, one may isolate and sequence either the full-length or partial 3′ end of the corresponding cDNA. To verify that the sequence fused to the transgene identified as above originated from genomic DNA next to the integration site, the sequence can be compared with a genomic DNA database of the host using a BLAST program and/or the genomic sequence next to the integration site can be isolated for direct sequencing and comparison with the isolated cDNA. Commonly used techniques to isolate genomic DNA next to an integration site include inverse PCR, ligation-mediated PCR, and randomly primed PCR or variations thereof. These techniques are known in the art and are described in Sorensen et al. 1999. Isolation of Unknown Flanking DNA by a Simple Two-Step Polymerase Chain Reaction Method. DYNALogue 3: 2-3; Cottage et al. 2001. Identification of DNA Sequences Flanking T-DNA Insertions by PCR-Walking. Plant Mol. Biol. Rep. 19:321-327; Yuanxin et al. 2003. T-linker-specific ligation PCR (T-linker PCR): an advanced PCR technique for chromosome walking or for isolation of tagged DNA ends. Nuc. Acid. Res. 31(12) e68; Zheng et al. 2001. Molecular characterization of transgenic shallots (Allium cepa L.) by adaptor ligation PCR (AL-PCR) and sequencing of genomic DNA flanking T-DNA borders. Transgenic Res. 10: 237-245; Spertini et al. 1999. Screening of Transgenic Plants by Amplification of Unknown Genomic DNA Flanking T-DNA. Biotechniques 27: 308-314; Liu et al. 1995. Efficient isolation and mapping of Arabidopsis thaliana T-DNA insert junctions by thermal asymmetric interlaced PCR. The Plant J. 8(3): 457-463; Ponce et al. 1998. Rapid discrimination of sequences flanking and within T-DNA insertions in the Arabidopsis genome. The Plant J. 14(4): 497-501.
(III) Transformation

Vector DNA can be introduced into prokaryotic or eukaryotic cells via conventional transformation or transfection techniques. The terms “transformation” and “transfection” are intended to refer to a variety of art-recognized techniques for introducing foreign nucleic acid (e.g., a transgene) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a transgene that encodes a selectable marker (e.g., resistance to antibiotics) is generally introduced into the host cells along with the gene of interest. Such selectable markers include those which confer resistance to drugs, such as G418, hygromycin and methotrexate. Nucleic acid encoding a selectable marker can be introduced into a host cell on the same vector as that encoding a transgene protein or can be introduced on a separate vector. Cells stably transfected with the introduced nucleic acid can be identified by drug selection.

In selecting a transformation vector, the host must be chosen that is compatible with it. In selecting an expression control sequence, a number of variables are considered. Among the important variables are the relative strength of the sequence (e.g. the ability to drive expression under various conditions), the ability to control the sequence's function, compatibility between the polynucleotide to be expressed and the control sequence (e.g. secondary structures are considered to avoid hairpin structures which prevent efficient transcription). Hosts are selected which are compatible with the selected vector, tolerant of any possible toxic effects of the expressed product, able to secrete the expressed product efficiently if such is desired, to be able to express the product in the desired conformation, to be easily scaled up, and to which ease of purification of the final product.

The choice of the expression cassette depends on the host system selected as well as the features desired for the expressed polypeptide. An expression cassette of the invention includes a promoter that is functional in the selected host system and can be constitutive or inducible. The expression cassette may also include a ribosome binding site; a start codon (ATG) if necessary; a region encoding a signal peptide, e.g., a lipidation signal peptide; a DNA molecule of the invention; and a stop codon. If the integrated DNA contains more than one cassette, a 3′ terminal region (translation and/or transcription terminator) may be part of the additional cassette. The signal peptide encoding region is adjacent to the polynucleotide of the invention and placed in proper reading frame. The signal peptide-encoding region is homologous or heterologous to the DNA molecule encoding the mature polypeptide and is compatible with the secretion apparatus of the host used for expression.

The open reading frame (transgene), solely or together with the signal peptide, is placed under the control of the promoter so that transcription and translation occur in the host system. Promoters and signal peptide encoding regions are widely known and available to those skilled in the art and include, for example, the promoter of Salmonella typhimurium (and derivatives) that is inducible by arabinose (promoter araB) and is functional in Gram-negative bacteria such as E. coli; the promoter of the gene of bacteriophage T7 encoding RNA polymerase, that is functional in a number of E. coli strains expressing T7 polymerase; OspA lipidation signal peptide; and RlpB lipidation signal peptide.

Expression cassettes constructed according to the present invention may contain sequences suitable for permitting integration of the transgene into the host genome. These might include transposon sequences, CRE-Lox and FLP recombination sequences, and the like, as well as Ti sequences which permit random insertion of a heterologous expression cassette into a plant genome.

The expression cassette(s) to be integrated into the host is typically part of a transformation vector. Vectors (e.g., plasmids or viral vectors) can be chosen, for example, from those known in the art. Suitable vectors can be purchased from various commercial sources.

Methods for transforming/transfecting host cells with expression vectors are well-known in the art and depend on the host system selected.

Upon expression, a recombinant polypeptide is produced and may remain in the intracellular compartment, secreted/excreted in the extracellular medium or in the periplasmic space, or embedded in the cellular membrane. The polypeptide is recovered in a substantially purified form from the cell extract or from the supernatant after centrifugation of the recombinant cell culture. Typically, a recombinant polypeptide is purified by antibody-based affinity purification or by other well-known methods that can be readily adapted by a person skilled in the art, such as fusion of the polynucleotide encoding the polypeptide or its derivative to a small affinity binding domain.

Numerous plant transformation vectors and methods for transforming plants are available. The selection of the vector depends on the preferred transformation technique and the target plant species to be transformed.

Methods for constructing plant expression cassettes and introducing transgenes into plants is generally described in the art. For example, methods for transgene delivery involve the use of Agrobacterium, PEG mediated protoplast transformation, electroporation, microinjection whiskers, and biolistics or microprojectile bombardment for direct DNA uptake. The method of transformation depends upon the plant cell to be transformed, stability of vectors used, expression level of gene products and other parameters.

The components of the expression cassette may be modified to increase expression of the inserted transgene. For example, the transgene may be modified for preferred codon usage in plants. DNA sequences for enhancing gene expression may also be used in the plant expression vectors. These include the introns of the maize Adhl, intronl gene, and leader sequences, (W-sequence) from the Tobacco Mosaic virus (TMV), Maize Chlorotic Mottle Virus and Alfalfa Mosaic Virus. The first intron from the shrunkent-1 locus of maize, has been shown to increase expression of genes in chimeric gene constructs.

Another approach to transforming plant cells with a heterologous gene involves propelling inert or biologically active particles at plant tissues and cells. This procedure involves propelling inert or biologically active particles at the cells under conditions effective to penetrate the outer surface of the cell in such manner as to incorporate the vectors into the interior of the cells. When inert particles are utilized, the vector can be introduced into the cell by coating the particles with the vector containing the transgene. Alternatively, the target cell can be surrounded by the vector so that the vector is carried into the cell by the wake of the particle. Other biologically active particles including dried yeast cells, dried bacteria, or bacteriophages, each containing the desired DNA, can also be propelled into plant cell tissue. In addition, the vectors of the invention can be constructed so that they are suitable for use in plastid transformation methods using standard techniques.

Bacteria from the genus Agrobacterium can be utilized to introduce foreign DNA and transform plant cells. Suitable species of such bacterium include Agrobacterium tumefaciens and Agrobacterium rhizogenes. A. tumefaciens (e.g., strains LBA4404 or EHA105) is particularly useful due to its well-known ability to transform plants.

Agrobacterium tumafaciens is a soil pathogenic bacterium that naturally infects wound sites and transfers its T-DNA to dicot and monocot angiosperm and gymnosperms. The genus Agrobacterium can transfer T-DNA to transform species from a broad kingdom range including fungi (yeasts, ascomycetes, and basidiomycetes) and human cells.

A general method of transforming and selecting plants for expression of a gene of interest was developed based on disarmed Ti-plasmids, leaf discs of tobacco, tomato or petunia, and selection of the regenerated transgenic plants using the antibiotic resistance conferred by the chimeric NOS-nptll-nos expression construct. A second expression cassette on the T-DNA binary vector plasmid also contained a gene of interest (nopaline synthase) which was expressed in the whole plant. Agrobacterium transformation methods have been used to introduce a variety of traits into numerous organisms including monocotyledonous and dicotyledonous plants, fungi and mammalian cells.

In its most basic form the T-DNA binary vector retains the functional features of two 25 base pair imperfect repeats known as the right and left borders (RB and LB, respectively) that define the boundaries of the T-DNA and encompass the gene of interest and all sequences necessary for expression of the gene in the host cell including the upstream promoter and downstream terminator. Expression of the vir genes on the Agrobacterium helper plasmid results in protein products that function in the excision, stabilization, translocation, and integration of the T-DNA into the host cell genome.

The RB and LB contain consensus nucleotide sequence cleavage sites recognized by the endonuclease VirD2 and VirE1 respectively that function to cleave the T-strand from the T-DNA vector. VirD2 also remains covalently attached to the RB conferring protection from further endonucleolytic digestion or degradation and facilitating translocation and subsequent integration of the T-strand into the host genome.

The transgene to be expressed according to the present invention may be any nucleic acid whose expression is desired. The transgene may encode polypeptides and structural RNAs. It may also encode anti-sense RNAs and ribozymes to degrade and/or inhibit translation of a target host-transcribed mRNA. The transgene may also be expressed to effect RNA interference of a target. RNA interference may be effected by having the transgene encode a precursor of short interfering RNAs (siRNA) or siRNA-like molecule.

The following example is presented as illustrative rather than by way of limitation.

### EXAMPLE

This example demonstrates the successful genetic integration of a structural gene in a host and the use of host sequences to facilitate the termination and polyadenylation of the transcript and subsequent gene expression. Genetic integration of the human IL-10 construct in the low alkaloid tobacco cultivar 81V9 (Menassa, R. et al. A self-contained system for the field production of plant recombinant interleukin-10.

Molecular Breeding. 2001 Sep; 8(2):177-185) in this example was accomplished with the binary vector form of Agrobacterium mediated transformation. The pORE_{—}04 parental T-DNA component binary vector used in this experiment is an improvement of pCB301 (Xiang. 1999. Plant Mol. Biol. 40:711-717) itself an improved version of the pBin19 plasmid, a hybrid derivative of the right and left borders of the nopaline TiT37 plasmid and the backbone of the broad host range plasmid pRK252 (Bevan, 1984. Nucleic Acids Res. 12:8711-8721). The plant selectable marker was neomycin phosphotransferase (nptII, Fraley, R. T. et al. Expression of bacterial genes in plant cells., editor. Proceedings of the National Academy of Sciences USA. 1983; 80(15):4803-4807; ISSN: 0027-8424).

The cloning of human IL-10 cDNA has been described previously (Menassa, R. et al. 2001, supra). The hIL-10 coding sequence was placed under the control of the enhanced 35S promoter of cauliflower mosaic virus (Kay, R. et al. Duplication of CaMV 35S promoter sequences creates a strong enhancer for plant genes. Science, USA. 1987; 236(4805):1299-1302) and the tCUP translational enhancer. This entire construct was directionally cloned into the pORE_{—}04 binary vector backbone (Accession # AY562542) using EcoR1 and SacII restriction enzymes. The final orientation of the hIL-10 coding sequence was such that the 3′ end of the gene was proximal to the right border and the direction of transcription initiated by the 35S promoter was in the 5′ to 3′ direction through the IL-10 coding sequence. Bioinformatic analysis using the WebGene HC_polyA software program was performed on the resulting binary vector expression construct to identify potential polyadenylation sites inclusive of the 3′ end of the gene and the location of the predicted right border cleavage site. No termination or polyadenylation sequences were found that would be predicted to prematurely terminate transcription of the IL-10 gene.

The binary vector transformation system was completed by transformation of an EHA105 Agrobacterium tumafaciens strain (Hood, et al. (1993) New Agrobacterium helper plasmids for gene transfer to plants. Transgenic Res. 2, 208-218.) with the binary vector. The transformed cells were grown on selection media containing 50 ug/ml kanamycin and 10 ug/ml rifampicin to maintain the plasmids. The low alkaloid tobacco cultivar 81V9 (Menassa et al. 2001 supra) was transformed by the leaf disc transformation method developed by Horsch et al. (1985. Science 227: 1229-1231). Whole leaves from greenhouse grown plants were sterilized by immersion in 70% ethanol for 1 minute, a brief rinse in sterile water, immersion in 10% bleach (containing 1 drop of Tween 20) for 5 minutes, followed by rinsing four times for 5 minutes in sterile water. Leaf tissue with the midvein excised was cut into 1 cm² fragments using a scalpel and incubated in an overnight culture of transformed Agrobacterium that had been centrifuged at 3000×g for 15 minutes for resuspension of the pellet in a 50% dilution of MST-1 media. The leaf discs were immersed in the Agrobacterium suspension for 30 seconds each side prior to being blotted briefly on Whatman No. 2 sterile filter paper to remove excess bacteria and placed epidermal side down onto MST-2 media containing 1 μg/ml BA and 0.098 μg/ml NAA but no selection at this point. The leaf discs were co-cultured with the Agrobacterium for 2 days in a 22° C. growth chamber on a 12 hour light cycle. To subsequently inhibit bacterial growth and initiate the selection of transformed tissues the explants were transferred to MST-3 media which in addition to the hormones also contains 500 ug/ml timentin and 100 ug/ml kanamycin and incubated at 22° C. for 2 weeks. Explants were transferred to new MST-3 media at 2 week intervals until callus development and shoots began to form at 3-5 weeks. Once well defined stems developed the shoots were excised and trimmed of all callus prior to being transferred to Magenta boxes containing MST-4 media.

Site-specific and predictable genetic integration of an exogenous gene into a defined location in complex polyploid genomes such as dicot tobacco plants is currently not possible. As such all current methods of genetic transformation require a screening procedure to select out undesirable and select for desirable genetic integration events (Kohli et al. 2003. Plant mol. Biol. 52: 247-258). Likewise, transformation of a host with our technology requires that undesirable genetic integration events including unpredictable recombination events are selected out with a systematic screening procedure as described below.

The first step is to ensure generation of a population of transformed hosts each member of which has arisen as result of an independent genetic transformation event. To ensure that each mature plant had arisen from an independent transformation event only one shoot from each explant is selected for further analysis. Regenerated plants are then grown under standard greenhouse conditions to maturity and selected for those which do not demonstrate undesirable phenotypic effects.

The second step in the screening procedure is to select for regenerated plants that contain a genetic insertion of the transgene of interest. This is accomplished through the isolation of genomic DNA and diagnostic polymerase chain reaction with primers specific to the transgene of interest.

One leaf disc representing (˜1 cm² or ˜10 mg) is subjected to lysis with plant PCR lysis buffer (200 mM Tris-HCl pH 7.5, 250 mM NaCl, 2.5 mM EDTA, and 0.5% SDS). The tissue was macerated in 400 ul of buffer using an electronic mini-drill and incubated at R.T. for 1 hour and subjected to centrifugation at 13,000 r.p.m. R.T. for 1 minute. 300 ul of the supernatant was aliquoted to new eppendorf tubes and DNA was precipitated with the addition of 300 ul of isopropanol, mixing and incubation at R.T. for 2 minutes. DNA was pelleted by centrifugation at 13,000 r.p.m. for 15 minutes. The supernatant was aspirated and discarded followed by washing of the pellet with 500 ul of 75% ethanol, vortexing briefly and spinning at 13,000 r.p.m. for 5 minutes. The supernatant was aspirated and discarded and the pellet allowed to air dry for 5-10 minutes prior to resuspension of the DNA pellet in 50 ul of sterile ddH2O. 3 ul of the resuspension was used as template in a PCR reaction to amplify the insert with primers specific to the 5′ (5′-CCCCTCCGCGGTGGTATGCACAGCTCAGCACTG-3′; SEQ ID NO:12) and 3′ (5′-GGGAATTCAGAGCTCGTCCTTGTGATGATGATGATGATGACCAGAAGAAGAACCGCGTGGCAC AAGGTTACGTATCTTCATTGTCAT-3′; SEQ ID NO:13) end of the IL-10 coding sequence. The thermocycler conditions were as follows: 94° C. 4 minutes, 30 cycles of 94° C. for 40 seconds, 55° C. for 40 seconds, 72° C. for 1 minute, and a final extension of 72° C. for 10 minutes. PCR amplified samples were subjected to 1% agarose gel electrophoresis and specific bands were visualized by the addition of ethidium bromide and illumination under ultraviolet light. Amplification of the expected ˜650 b.p. band in transformed plants not present in the control non-transformed 81V9 tissue is indicative of a positive transformant containing the IL-10 coding sequence. In this example, of the 46 transgenic plants generated 23 were positive for the presence of the IL-10 coding sequence.

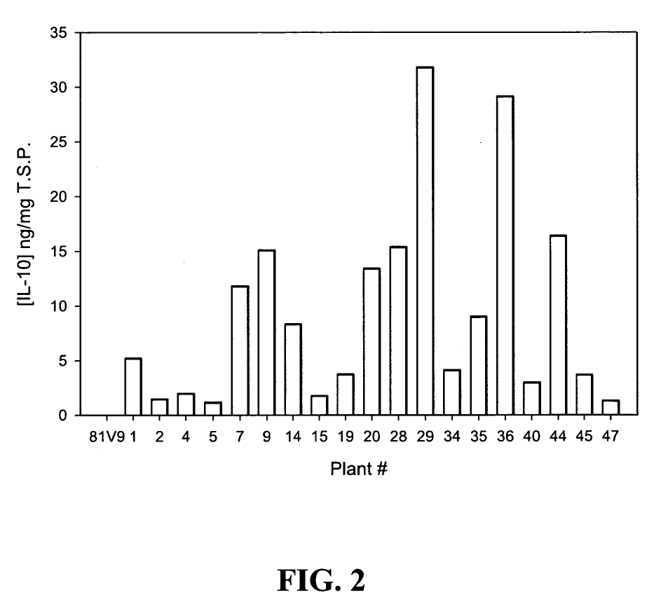

As the objective in most instances is the expression of the specific protein associated with the introduced gene positive transformants are further selected on this criteria. This is accomplished with a screening test in which total soluble protein is isolated from a positive transformant and qualitatively or quantitatively assessed by the ELISA technique. Plants were grown in greenhouse conditions to approximately the eight leaf stage at which point 3 whole leaf samples representing the top, middle and bottom of the plant were collected and frozen at −80° C. for later analysis. ˜0.3 g of leaf tissue was ground in a 3 x volume of protein extraction buffer (1×PBS, 0.05% Tween 20, 2% PVPP, 1 mM EDTA, 1 mM PMSF, 1 ug/ml leupeptin) using a mortar and pestle. The ground material was transferred to an eppendorf tube and centrifuged at 4° C. for 15 minutes at 14,000 r.p.m. to pellet the plant material. The supernatant was transferred to a new eppendorf tube and stored on ice for immediate use or at −80° C. for subsequent analysis. For the cytokine ELISA, anti-IL-10 antibody was diluted to 2 ug/ml in binding solution (0.1 M Na2HPO4 pH 9.0) and 50 ul was added to the wells of a 96 well enhanced protein binding ELISA plate (Nunc Maxisorb) for incubation at 40C overnight. The following day, the plates were washed 4 times with 200 ul PBS/Tween (1×PBS/0.05% Tween 20) and non-specific binding was blocked by incubation of 200 ul/well of 1% BSA in PBS for 30 minutes. The wells were washed 3 times with 200 ul of PBS/Tween and recombinant IL-10 standards and test samples were diluted in Blocking Buffer/Tween (PBS/Tween+1% BSA) and added to the wells for incubation at 4° C. overnight. The following day, the wells were washed 4 times with 200 ul of PBS/Tween and IL-10 was detected by the addition of a biotinylated anti-IL-10 antibody diluted to 1 ug/ml in Blocking buffer/Tween and added at 100 ul/well for incubation at R.T. for 1 hour. The wells were washed 6 times with PBS/Tween and detection was facilitated by the addition of 100 ul to each well of avidin-peroxidase diluted 1:2500 in Blocking Buffer/Tween and incubated for 30 minutes at R.T. The wells were washed 8 times with PBS/Tween and detection carried out by addition of 100 ul of ABTS substrate solution to each well and incubation at room temperature 5-60 minutes for sufficient colour development. The optical density was read at 405 nm and concentration of IL-10 in the samples was determined relative standards prepared on the same plate. IL-10 concentration was normalized to protein concentration as determined by Biorad assays performed on identical extract preparations. In this example, of the 23 transgenic plants PCR-positive for the IL-10 coding sequence, 19 were found to accumulate IL-10 protein (FIG. 2) and had no undesirable phenotypic effects resulting from transgene insertion.

As with other transformation methods it is expected that within a population of host cells there will be a range of protein expression as a result of unique genetic integration events in each host. The precise location of the genetic integration of the gene into the host genome can have effects on introduced gene expression resulting from position effects due to local contextual features such as chromatin organization. Further, our method relies on the acquisition of host encoded polyadenylation signals that when transcriptionally fused to the transgene of interest function as termination/polyadenylation signals and as such each integration event will exhibit different regulatory effects depending upon the location of integration and the sequence that becomes transcriptionally fused to the introduced gene. In the case of Agrobacterium transformation using binary and co-integrative vectors there is a vast literature demonstrating that in any population of transformed host cells there will be a percentage of transformants with undesirable T-DNA integration events including multiple insertions, concatomers, inverted and direct repeats, partial T-DNA deletions, binary vector or T-DNA recombination and insertion events, etc. These undesirable genetic insertion events can also be selected out when generating a host cell to express the introduced gene in the desired manner. In some cases, expression of the protein of interest with no observable undesirable phenotypic effects on the host may be all that is required. If the demands of the intended application warrant further screening the undesirable genetic events can be selected out in a number of ways. A commonly used technique to identify transformation events in which one T-DNA copy has been inserted into the host genome is with the technique of southern analysis.

All of 19 of the IL-10 expressing phenotypically normal transgenic plants were chosen for further analysis. To confirm that the introduced terminatorless gene is expressed as a transcriptional fusion with host encoded genomic DNA a modification of the 3′ rapid amplification of cDNA (3′ RACE) technique was performed. Sequencing of the resulting products allows identification and characterization of the sequences transcriptionally fused to the transgene. In addition, in host genomes in which sequence data is available, the location of the genetic insertion may be pinpointed by using the identified transcriptionally fused sequence as a reference point for searching the host genome database. Total RNA was isolated from plant tissue with the QIAGEN RNeasy kit according to the manufacturer's recommendations for plant tissue and on-column DNaseI treatment. RNA was eluted from the spin columns with 160 ul of DEPC-treated sterile water and stored on ice for immediate use or at −80° C. for subsequent analysis. First strand cDNA synthesis was carried out according to the Ambion RLM 3′ RACE protocol according to the manufacturer's recommendations. The reactions were incubated at 42° C. for 1 hour and placed into −20° C. for subsequent analysis. 1 ul of the RT reaction was used as template in the first PCR amplification to amplify specific IL-10 transcripts. Platinum Taq High Fidelity DNA polymerase was used to amplify via PCR specific products with the 3′ RACE Outer primer and a 5′ biotinylated IL-10 gene specific primer 2 (5′-CCCAAGCGAGAACCAAGAC-3′; SEQ ID NO:14). The resulting PCR products were purified using streptavidin coated magnetic beads (Dynabead M-280) according to the manufacturer's recommendations. Briefly, amplified biotinylated PCR fragments from the first PCR were isolated by mixing 40 ul of the PCR reaction with 40 ul of 200 ng of prewashed streptavidin coated magnetic beads and incubating for 15 minutes at R.T. After washing in 1×B&W buffer the bound double stranded biotinylated DNA is denatured by addition of 8 ul of 0.1 M NaOH and incubation for 10 minutes at R.T. The supernatant containing the non-biotinylated DNA strands was collected and neutralized with 4 ul of 0.2 M HCl and 1 ul of 1 M Tris-HCl pH 8.0. The sample volume was adjusted to 30 ul with 10 mm Tris-HCl pH 8.0 and 2 ul was used as a template in a second PCR reaction using the 3′ RACE inner primer nested gene-specific IL-10 primer3 and a biotinylated primer to the constant end of the 3′ RACE inner primer (5′-CGCGGATCCGAATTAATACGACTCACTATAGG-3′; SEQ ID NO:15). The PCR products were resolved on 1% agarose gel electrophoresis and specific bands were visualized by the addition of ethidium bromide and illumination under ultraviolet light. Bands were excised from the gel and purified with GeneClean gel extraction kit and eluted with 15 ul of elution buffer. The purified products were sequenced directly with a further nested IL-10 specific primer 4 (5′-AAGCTCCAAGAGAAAGGCATC-3′; SEQ ID NO:16).

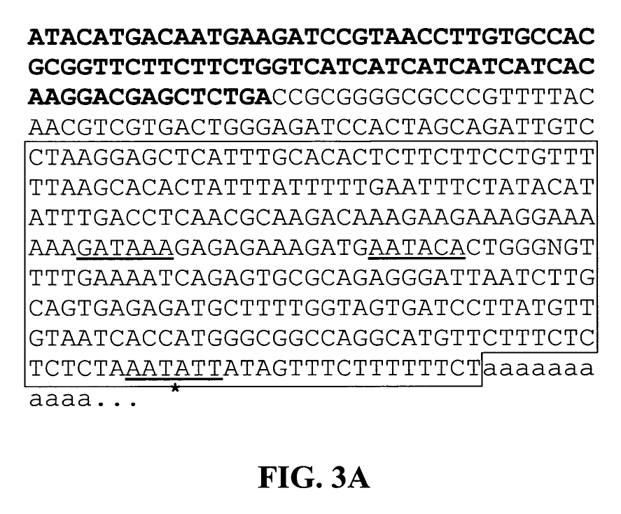

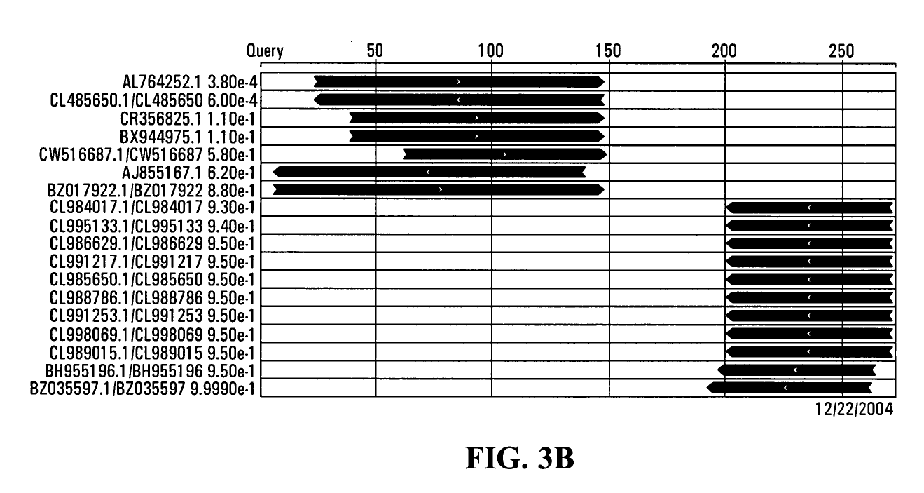

Sequence analysis of the partial cDNA allows identification of host sequence that is transcriptionally fused to the 3′ end of the integrated IL-10 coding sequence. As the tobacco genome has not been sequenced this sequence was compared to Higher Plant BACEND sequences (GSS sequences in GenBank 2.2.10) using WU-BLAST 2.0 located at the Arabidopsis Information Resource website to verify its plant origin (http://www.arabidopsis.org/wublast/). This sequence was also analyzed for the presence of poly A sites as identified by the HC_POLYA program. FIG. 3 illustrates a representative example from Plant 14 that demonstrates highly homologous plant sequence (3B) isolated from the Plant 14 tobacco genome as a transcriptional fusion with the genetically integrated IL-10 coding sequence (3A). This tobacco genomic sequence also contains poly A sites within the accepted range of 10-40 base pairs of the start of the poly A tail that resulted in transcriptional termination of the IL-10 coding sequence-tobacco genomic sequence transcriptional chimera and subsequent IL-10 gene expression (FIG. 2).

1. An expression cassette, comprising a promoter operably linked to a transgene which encodes a polypeptide consisting of an amino acid sequence, such that when the expression cassette is integrated in a host cell and the transgene is transcribed, transcription of the transgene terminates at a non-coding region in the genome of the host cell and not at a sequence within the cassette, and the polypeptide consisting of the amino acid sequence is expressed from the transcript.

2. The expression cassette according to claim 1 wherein, when the transgene is transcribed, the resulting RNA transcript comprises non-coding sequence from the host cell at the 3′ end, and the cassette-derived sequence in the RNA transcript is contiguous at the 3′ end with the non-coding sequence from the host cell.

3. The expression cassette according to claim 1 which is free of potential transcription termination sites in the region 3′ of the transgene.

4. The expression cassette according to claim 3 wherein the host cell is a plant cell and wherein the expression cassette is free of the following potential transcription termination sites in the region 3′ of the transgene:
AACAAA, AATAAA, AATAAC, AATAAG, AATAAT, AATACA,
AATAGA, AATATA, AATATT, AATTAA, ACTAAA, AGTAAA,
ATTAAA, CATAAA, GATAAA, GATTAA, AATGGA, AATGAA,
AATCAA, AAAAAA, AAGAAA, AATCAA, and TATAAA.

5. The expression cassette according to claim 1 wherein the non-coding region is an intergenic region of the genome, an intronic region of a gene within the genome, or a regulatory region of a gene within the genome.

6. The expression cassette according to claim 1 wherein the transgene encodes a recombinant protein which is other than a selectable marker or a reporter.

7. The expression cassette according to claim 1 further comprising a far upstream enhancer (FUE) sequence 3′ of the transgene.

8. A transformation vector comprising the expression cassette according to claim 1.

9. The transformation vector according to claim 8 further comprising a selectable marker gene.

10. A non-human organism having stably integrated in its genome the expression cassette of claim 1.

11. A method for expressing a transgene in a host cell, wherein the transgene encodes a polypeptide consisting of an amino acid sequence, the method comprising the steps of:
a) stably integrating into the host cell genome an expression cassette, wherein the expression cassette comprises a promoter functional in the host cell operably linked to the transgene such that, when the expression cassette is integrated and the transgene is transcribed, transcription of the transgene terminates at a non-coding region in the host cell genome and not at a sequence within the cassette; and
b) culturing the host cell comprising the expression cassette to express the transgene and obtain the polypeptide consisting of the amino acid sequence.

12. The method according to claim 11 wherein, in step (a), the expression cassette is integrated in a non-coding region of the host cell.

13. The method according to claim 11 wherein, when the transgene is transcribed, the resulting RNA transcript comprises non-coding sequence from the host cell at the 3′ end, and the cassette-derived sequence in the RNA transcript is contiguous at the 3′ end with the non-coding sequence from the host cell.

14. The method according to claim 11 wherein the expression cassette is free of potential transcription termination site in the region 3′ of the transgene.

15. The method according to claim 14 wherein the host cell is a plant cell and wherein the expression cassette is free of the following potential transcription termination sites in the region 3′ of the transgene:
AACAAA, AATAAA, AATAAC, AATAAG, AATAAT, AATACA,
AATAGA, AATATA, AATATT, AATTAA, ACTAAA, AGTAAA,
ATTAAA, CATAAA, GATAAA, GATTAA, AATGGA, AATGAA,
AATCAA, AAAAAA, AAGAAA, AATCAA, and TATAAA.

16. The method according to claim 11 wherein the non-coding region is an intergenic region of the genome, an intronic region of a gene within the genome, or a regulatory region of a gene within the genome.

17. The method according to claim 11 wherein the transgene encodes a recombinant protein which is other than a selectable marker or a reporter.

18. The method according to claim 11 wherein the expression cassette further comprises a far upstream enhancer (FUE) sequence 3′ of the transgene.

19. A method for expressing a transgene in a host cell, the method comprising the steps of:
a) transforming the host cell with the transformation vector of claim 8 such that the expression cassette is stably integrated into the host cell genome; and
b) culturing the host cell obtained from step (a) to express the transgene and obtain the polypeptide consisting of the amino acid sequence.

20. The method according to claim 11 wherein the host cell is a plant cell, an animal cell, or a fungal cell.

21. The transformation vector according to claim 8 which is an Agrobacterium vector.

22. The organism according to claim 10 which is a plant.

23. The organism according to claim 22 which is a dicot plant.

24. The organism according to claim 22 which is a monocot plant.

25. The organism according to claim 10 which is an animal.

26. The organism according to claim 10 which is a fungus.

27. The organism according to claim 10 which is a yeast.

28. The method according to claim 11 wherein the host cell is a dicot plant cell.

29. The method according to claim 11 wherein the host cell is a monocot plant cell.

30. The method according to claim 11 wherein the host cell is a fungal cell.

31. The method according to claim 30 wherein the fungal cell is a yeast cell.

32. The method according to claim 11 wherein the host cell is an animal cell.
